(19) United States
(12) Patent Application Publication
(10) Pub. No.: US 2008/0142021 A1
(43) Pub. Date: Jun. 19, 2008

(54) Condom

(76) Inventor: Lamont Van Hook, Lancaster, CA (US)

Correspondence Address: LAW OFFICES OF CLEMENT CHENG, 17220 NEWHOPE STREET #127, FOUNTAIN VALLEY, CA, 92708, US

(21) Appl. No.: 11/641,227

(22) Filed: Dec. 19, 2006

(51) Int. Cl. A61F 6/04

(52) U.S. Cl. 128/844

(57) ABSTRACT

A multi-function measurement condom includes an indicia that enables the user to accurately measure his penis in its erect state, while simultaneously aiding in the prevention of pregnancy and sexually transmitted diseases. The condom is comprised of a tubular-shaped sheath with at least an inner layer and an outer layer of an elastic material, a dome-shaped closed end, and an opposite open end. A protrusion on the sheath extends lengthwise, and an indicia is placed between the inner and outer layers of the sheath. This creates a dual length unit with two different scale marks positioned adjacently, and the indicia extending centrally over the elongated protrusion. The indicia are visible through the outer layer so that the proper measurement can be deciphered by an individual. A niche located on the inner layer of the sheath incorporates the long protrusion into the rest of the apparatus smoothly, and it extends from the tip of the condom to a point short of its open end.

### BACKGROUND OF THE INVENTION

A. Field of the Invention

The present invention relates to a contraceptive device. More particularly, the present invention relates to a condom with a longitudinal erectometer for telling the current length of the male sexual organ precisely.

B. Description of the Prior Art

It is extremely beneficial to diagnose serious medical conditions such as prostatitis, diabetes, cardiovascular disorders, and cerebrovascular disorders in the earliest phases of onset. However, the reality is that these diseases often exist within a patient for an extended period of time before he seeks medical attention from a licensed practitioner. This is partially due to the fact that men would rather avoid an uncomfortable physical exam unless there is a perceived serious problem at hand. The present invention was created to remedy this obstacle in disease prevention and early diagnosis. Because it is difficult to measure the size of an erection by the naked eye alone, the condom measuring device allows a man to occasionally check his penis for erectile consistency. If the man notices a slight discrepancy in size, he can closely observe his prostate for any further signs of abnormality. Since most of the aforementioned disorders are associated with a gradual decline of erection size, the man is more likely to visit a licensed physician and obtain a professional opinion on the matter if he is aware of a declining erection size. The condom device, in effect, can lead to the early diagnosis and treatment of prostate problems when used properly.

Throughout his sexual life, it is important that a man is aware of his ability to get and maintain an erection, it can even be recommended that he keep a record of his erectile history. The accurate comparison of erection size over time can serve as a key indicator of a man's overall physical health. With this information, he can modify or maintain his current lifestyle with regard to an exercise routine and other healthy habits.

Previous methods employed to create an accurate record of erection size involve attempts to apply a measuring tape or ruler to the sensitive genital area. Even if this proves an effective system of measurement, it is extremely awkward. Furthermore, pressing the cold and sharp edge of a stick or steel tape against body tissue is both unpleasant and unsanitary to the body. A vinyl measuring tape may seem to be a decent alternative, however it does not allow for a one-handed measurement.

There is currently a condom marketed in the United States and online named the “Peter Meter”, yet its packaging and advertisement clearly convey the message that it is “Sold as a Novelty Item Only.” The unapproved contraceptive device has proprietary length marks printed longitudinally on the condom. The printed marks are visible on either side of the condom-shaped layer, in a matter wherein they are easily stretched if the device were tightening around the penis. It is important to note that no means or suggestions as to how to take an accurate measurement while preventing or compensating for the change in distances between printed markings are included on the device.

It is widely accepted that the materials that are used to create condoms, or other any contraceptive device, should be carefully selected and processed in medically adapted facilities. It is also imperative that a condom does not transfer any semen to the bodily orifices of a sexual partner, particularly the mucous membrane, vaginal walls, and cervix of a female. Such transfers can lead to an accidental conception; thus, every effort should be made to keep the condom as safe as possible to avoid possible health complications. It is because of the aforementioned reasons that a method must be developed to protect the sensitive female reproductive areas from direct contact with printed markings on condoms.

In addition, the accuracy of measurement displayed by the marks is integral to the dependability of the condom to provide a precise reading. Therefore, the condom must be adapted to limit the natural stretching which typically occurs during use. This provision has not yet been addressed by prior prototypes.

The object of the present invention is to provide a multi-function measurement condom that does not pose any health hazards to its customers.

Another object of the present invention is to provide a condom with a safe and accurate measurement of a male erection that coincides with the traditional contraceptive function of a condom; this allows for periodic measurement of personal potency through routine condom usage.

Yet another object of the present invention is to provide a multi-function measurement condom indicates erection length and provides a remedy for unintentional slipping or unrolling of the condom. This is achieved by adding an additional stiffening device to the condom walls without substantially affecting the material elasticity of a normal condom.

Yet another object of the present invention is to provide a multi-function measurement condom that can be hidden from the naked eye by simply placing the radial position of the measuring device on the bottom side of the penis. This also serves to prolong the duration before ejaculation by increasing the local constriction.

Yet another object of the present invention is to integrate the steps of creating a multi-function measurement condom to the conventional condom manufacturing process, without requiring a significant change in existing condom facilities.

### SUMMARY OF THE INVENTION

A condom, according to the present invention, has an integrated erectometer which functions to measure the longitude of a penile erection with additional contraceptive protection. The condom is comprised of a tubular-shaped sheath with at least an inner layer and an outer layer of an elastic material, a dome-shaped closed end, and an opposite open end. A long protrusion on the sheath extends lengthwise, and an indicia is placed between the inner and outer layers of the sheath. This creates a dual length unit with two different scale marks positioned adjacently, and the indicia extending centrally over the elongated protrusion. The indicia are visible through the outer layer so that the proper measurement can be deciphered by an individual.

The lengthwise protrusion on the sheath is formed by a complementary niche on the inner layer of the sheath, which extends from the dome to a point just short of the open end. It is positioned toward the central axis of the tubular sheath.

The indicia are formed, in part, by depositing resin in an embossed form of a ruler, which lies on a circumferential section of the inner layer of the sheath; the lengthwise protrusion on the sheath is formed by a raised area of the outer layer that extends away from the sheath's central axis.

The lengthwise protrusion on the sheath may formed by the combination of the inner layer niche on the central axis of the sheath and the raised area of the outer layer that extends away from the central axis of the sheath. The protrusion begins at the dome and ends at a point just short of the open end of the condom. The indicia are formed, in part, by depositing resin in an embossed form of a ruler over the inner layer niche. The resin may be silicon, a component that has the ability to be externally inked; this serves to provide a contrast between the concave and convex regions of the embossed form of a ruler. The sheath may be transparent or semitransparently colored in both primary and non-primary shades. The condom is also comprised of locally extended sheath walls with ribbed or studded regions along the internal and external surfaces of the condom.

One common method of forming the indicia is to use ink for printing a starting mark, type of measurement unit, centerline, scales and numerical hash marks that indicate length. Units can be defined in the English system of measurement in inches and/or the Metric system of measurement in centimeters. In order to produce the indicia, a rotary press station may be added to an automatic condom manufacturing facility. There are several additional methods by which indicia can be printed, which include a rotogravure press, a flexography, screen printing, laser printing, and inkjet printing.

The purpose of the indicia is extended by a printed volume scale on the dome of the sheath, which measures the amount of ejaculate released in a single session.

The lengthwise protrusion on the sheath walls provides stiff support, which serves to isolate the indicia from potential changes in the shape or dimensions of the sheath. This serves to minimize the elasticity, and ultimately maintain accuracy of the length measurement even with the manipulation of the condom over the erect penis. An added benefit of the inner condom layer niche and indicia combination is a local constriction, which the user may selectively apply to his penis in order to suppress ejaculation and prolong the erection.

A condom, according to another embodiment of the present invention, has an erectometer for measuring the length of a penile erection under contraceptive protection. The condom is comprised of a tubular-shaped sheath with at least an inner layer and an outer layer of an elastic material, a dome-shaped closed end, and an opposite open end. A long protrusion on the sheath extends lengthwise, and the indicia are placed between the inner and outer layers of the sheath. This creates a dual length unit with two different scale marks positioned adjacently, and the indicia extending centrally over the lengthwise protrusion. The indicia are visible through the outer layer so that the proper measurement can be deciphered by an individual.

The lengthwise protrusion on the sheath is created by a partial withdrawal of the inner layer toward central axis of the sheath. This creates flattened wall section that relative to the wall areas of the rest of the inner layer, and the flattened wall section extends from the dome of the condom to a point just short of the open end. The ruler insert provides isolation for the indicia from any potential misshapenness resulting from the elastic nature of condoms. This configuration allows for an accurate penile measurement, even during the application of the condom over the erect penis.

The combination of the aforementioned inner condom layer's flattened wall section and the ruler insert provides a local constriction which enables the user to selectively suppress the ejaculation and prolong the erection time.

A condom, according to yet another embodiment of the present invention, has an erectometer for measuring the length of a penile erection while under contraceptive protection. The condom is comprised of a tubular-shaped sheath with at least an inner layer and an outer layer of an elastic material, a dome-shaped closed end, and an opposite open end. A long protrusion on the sheath extends lengthwise, and the indicia are placed between the inner and outer layers of the sheath. This creates a dual length unit with two different scale marks positioned adjacently, and the indicia extending centrally over the lengthwise protrusion. The indicia are visible through the outer layer. The condoms, according to the present invention, also have an extra anti-slip feature.

The rigid region, which results from the niche and ruler insert, enhances the constriction of the condom around the penis. This prevents the condom from slipping off prematurely (even in its flaccid state), which frequently occurs before and after the full erection of the penis.

Furthermore, during operation of the condom the layer thickness of the scale indicia stiffens the condom walls against a longitudinal compression to prevent a collapse of the condom that may cause it to slip away and a possible failure of contraception.

Embodiments of the invention will now be described by way of example with reference to the accompanying drawings.

### BRIEF DESCRIPTION OF THE DRAWINGS

FIG. 1 is a partially broken away perspective view of a condom erectometer according to a first embodiment of the present invention.

FIG. 2 is a cross-sectional view of the condom erectometer taken along line 2-2 of FIG. 1.

FIG. 3 is a longitudinal cross-sectional view of the condom erectometer taken along line 3-3 of FIG. 1 showing a fold that holds the ruler indicia of an ink layer protruding inwardly of the condom.

FIG. 4 is a longitudinal cross-sectional view of a condom erectometer of a second embodiment with an outwardly protruding wall area embossed to provide three-dimensional ruler indicia on the condom surface.

FIG. 5 is a partially broken away perspective view of a condom erectometer according to a third embodiment of the present invention.

FIG. 6 is a perspective view of a film ruler for molding into the condom erectometer of FIG. 5.

FIG. 7 is a cross-sectional view of the condom erectometer taken along line 7-7 of FIG. 5.

FIG. 8 is a perspective view of a condom erectometer according to a fourth embodiment of the present invention.

FIG. 9 is a cross-sectional view of the condom erectometer taken along line 9-9 of FIG. 8.

FIG. 10 is a side elevational view of the condom erectometer of FIG. 8.

FIG. 11 is a plan view of the condom erectometer of FIG. 8 as seen normally during its operation.

### Similar reference numbers denote corresponding features throughout the attached drawings.

### DETAILED DESCRIPTION OF THE PREFERRED EMBODIMENT

In reference to FIG. 1, a condom 10 of the present invention is comprised of a sheath made of an elastic material (i.e. conventional latex) or a synthetic material (i.e. polyurethane) which can be used to shape the condom by either dipping or using on a rod shaped mandrel. Proper treatment of the latex material and dipping operation of the mandrel are well established in the art, and they will not be elaborated upon in the present invention.

U.S. Pat. Nos. 4,428,385; 4,960,131; 5,377,691 and 5,779,621 outline erection measurement devices that measure the girth of the penis and do not incorporate a contraceptive element.

The inventor was unable to find a condom similar to the present invention disclosed in any earlier patent literature.

The condom 10, according to the present invention, has all of the same the features of traditional reliable condoms, which are generally accepted as the best available method of contraception and sexually transmitted disease prevention.

A condom 10, in its most general tubular shape, is sealed on the front end by a shaped dome 12 with a tip 14, which provides adequate space within the condom for the male ejaculate. The other end of the condom 10 has an annular opening 16 which is formed by a ringed convolution 18 of condom walls. The walls help secure the condom 10 to the penis by constricting the base of the penis. The convolution of walls also serves to permit the rolling and unrolling of the condom 10, which is necessary to transform the initial, compressed form of the condom to extend over an erect penis. The condom 10 may be transparent or semitransparently colored with shades of reds, yellows, browns, purples, greens, blues, et cetera. Exotic colors and patterns may also be used for the condom 10.

The erectometer of the present invention can also be incorporated into non-conventional types of condoms, such as a “baggy fit” style condom, which has a tight base ring and locally extended walls with smooth, ribbed or studded regions along internal and/or external surfaces of the condom.

According to the present invention, the condom erectometer can be created by the traditional dipping processes the installation an additional printing plate to appropriate location on the machinery. A conveyer belt can transfer the initial dipped forms to the respective mandrels in a timely manner.

If the condom 10 is to be comprised of a liquid thermoplastic polyurethane material, a condom-shaped mandrel is typically first dipped into a polyurethane solution at a rate of approximately 16 to 90 centimeters per minute; this allows for the film to assume the shape of the mandrel. Before withdrawal, the condom form may be left in the solution for approximately 20 to 70 seconds so that the polyurethane film can air-dry. Conventionally, the condom film may go through a subsequent second dipping process in order to achieve a desired wall thickness. The condom form is then cured at elevated temperature of about 130 to 175 degrees Centigrade (Celsius) for about 20 to about 40 minutes.

As a result of this procedure, the condom 10 gains enough elasticity to readily stretch and conform to the profile of the penis. Thus, the condom 10 in operation distends slightly more at the dome 12 areas than the rest of the condom 10. In addition, the condom 10 is generally round in circumference with an area of its top surface 22 embedded with ruler indicia 20. The indicia 20 extend lengthwise along the top surface 22 from the rear ring 16 to the front dome 12. The ruler indicia 20 may be created on the condom by known printing methods during manufacture, as described below. Also printed on condom 10 is an optional scale of volume 11 indicated in cubic centimeters, which may be inked on the dome area 12 of the condom. The purpose of this measurement is to indicate the amount of ejaculate, as variations in a man's amount of ejaculate can serve as an indication of disease or fertility abnormality.

In order comfortably include the indicia 20 into the condom 10, a niche 24 is formed in the condom 10 behind the dome 12 and preferably stops short of where the glands penis will be lodged. This is so that the niche 24 lies flat on the top surface of the shaft of penis. During the actual use of the condom 10, the erectometer may be hidden from plain sight by turning the indicia 20 along with the niche 24 to the other side of the penis shaft of the penis. Regardless of the indicia's location, the device still provides reliable contraception

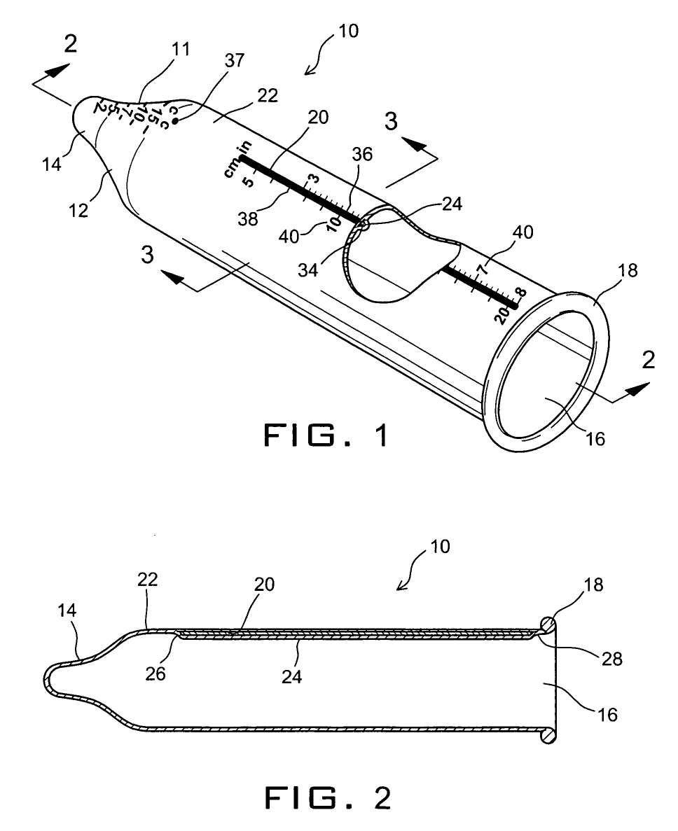

The profile of the niche 24 can be further explicated by the sectional view of FIG. 2. The niche 24 may be comprised of a groove made on the mandrel. If the mandrel were made of glass, the groove may be made by a cylindrical mandrel that is heat-treated in order to smooth out any burrs or sharp edges.

The niche 24 is strategically slanted at a front end 26 toward the condom walls 22 in order to connect the condom walls to the walls of niche 24 in a solid line. In addition, the opposite end 28 of the niche 24 at the rear is located in front of the ring 18 and may be shaped in congruently to the front end 26. Slacks from the opposite ends 28, 30 absorb the stretching of the condom walls during its initial manipulation over the penis, and this allows for an accurate, readable length.

The aforementioned niche 24 of the condom 10 also functions to isolate of the indicia 20 from the possible dimensional changes in surface area of the condom 10 due to its elasticity.

The niche 24 has the indicia 20 of a double-sided scale. Thus, the length marks may indicate the English system of measurement 36 from the zero point 37 to eight or more inches on one side, and the Metric system of measurement 38 from the zero point 37 to twenty centimeters, extending in parallel with the opposite inch marks 36. These scale marks are joined by a thicker and wider centerline 34. Numerals 40 may be printed in close proximity to their corresponding scale marks. The measurement markings “cm” and “in” may be also printed to distinguish between the two different unit systems.

According to the present invention, the condom 10, during its initial mandrel dipping processes, passes through a printing stage between the two dipping stages. During this phase, the condom-shaped mandrel is fully immersed in a bath of latex (or the like) and then subject to a drying time interval; this creates a predetermined thickness of the sheath. After the sheath is cured, it is peeled from the mandrel, carefully tested for possible pores, and packaged in a rolled form for consumer use. Before the condom 10 gets to the final dipping stage, the use of a printing press (i.e. rotary) can be integrated into the process (not shown).

The rotary press is type of printing press wherein the impressions are curved around a cylinder so that printing can be done on continual basis to a supply of condom sub layers on mandrels. Types of rotary presses used to print the scale lines on the condom can be offset presses or rotogravure presses. However, printing processes applicable to the present invention are not limited to the above examples; other available methods of printing may be applied, such as offset lithography, gravure, flexography, screen, laser or inkjet printing. In addition to or in the absence of printing ink, the top surface 22 of the condom 10 may be creped or embossed with the pattern of a ruler. This can occur in one continuous operation, either during or after dipping, is performed in printing on substrates of paper towels, cards and other similarly manufactured products.

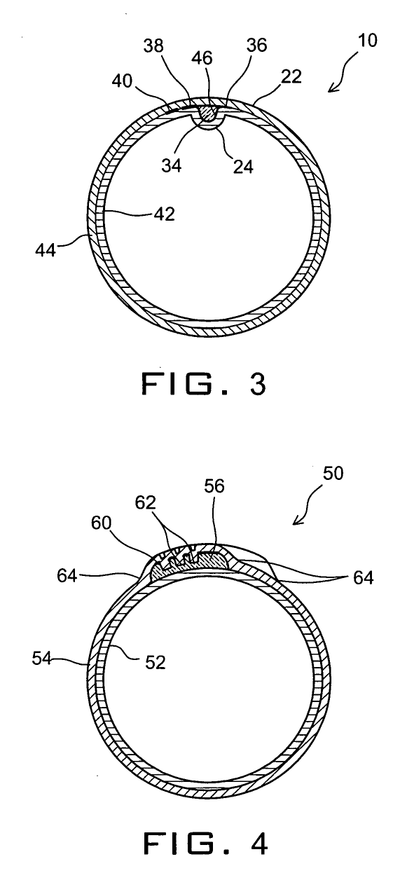

The transitions of the niche 24 into the surrounding condom walls are further explicated by the cross-sectional view of FIG. 3. The condom walls may comprise an inner layer 42 and an outer layer 44. The niche 24 may extend into the inner layer 42 and provide a trough 46 facing outwardly of the condom 10 to hold an ink deposit for the straight centerline 34 of the indicia 20. The indicia 20 protrude toward the penis of the condom wearer. Scales 36, 38 and length marks 40 are printed onto either side of the centerline 34 at the general plane of the condom 10. The outer layer 44 provides a protective covering over the indicia 20 during the second dipping process; this isolates the ruler indicia 20 into the body of the condom. The selection of darker ink for indicia 20 appropriately contrasts to the bright substrate color of the overall condom 10, which allows for the indicia 20 to be highly visible to the condom wearer. The complete separation of the indicia 20 from the human skin ensures a sterile contraceptive device that will never transfer ink to the genital area of a sexual partner.

The slightly thickened section of the condom 10 functions to constrict the condom 10 without placing excessive pressure on the penis. Due to the concentration of materials about the niche 24, the condom 10 becomes more rigid lengthwise. Thus, a longitudinal pull on the condom does not substantially stretch the ruler at the centerline 34 and the scales 38 attached to the centerline 34, thereby maintaining the precision of measurement.

An added benefit of having a lightly smaller condom diameter at the niche 24 is evident if when erectometer is hidden from plain sight. This is achieved by placing the indicia 20 along with niche 24 on the bottom side of the penis, which is the Corpus Cavernosum Urethrae, wherein ejaculation occurs. The extra constriction by the niche 24 at the right spot helps prevent premature ejaculation.

In addition, the partially thickened condom 10 will resist bunching up and the inadvertent unrolling of its walls during use. It also helps to keep the condom securely on the penis, which may increase the contraception success rate.

FIG. 4 shows an alternative embodiment, wherein the condom 50 is composed of an inner layer 52 and an outer layer 54. Lodged between the two layers 52 and 54 is an embossed ruler 56.

A conventional, smooth-surfaced mandrel can be used to create the first layer 52, which is then left to dry. The inner layer 52 typically has a smooth annular surface, on which the ruler form 56 is printed using ink and/or an alternative colored resin material. There are different ways to form the embossed ruler 56 on the layer 52.

In an “ink-first” method of embossing, the printing ink may be used create the ruler image on the exterior of the form 52, as shown in FIG. 1. Another method, offset printing, involves first transferring the print onto a durable rubber pad. The layer 42 is then pressed or rolled over the print, causing the ink to transfer to the desired location. Latex and thermoplastic polyurethane, the most common condom 50 materials, are non-porous by nature, which allows ample time for the application of embossing powder on the wet ink. After removing the excess, the existing powder is melted into a raised ruler 52 design by means of the residual heat of the condom layer 52. Embossing powders are available in various brands and colors, each with different melting points. The color, opacity, or gloss of each powder type allows for wide range of diverse styles.

Silicon coatings may also be used to create the raised ruler 56 instead of ink and powder. Silicon has previously been used in both the fields of medical instrumentation and graphic printing. For example, silicon is utilized to print logos or patterns of clothing garments in order to provide a soft, non-slip grip. This concept is illustrated in U.S. Pat. No. D455,014 to Cheynet, which shows a fabric ribbon with a shaped silicon coating.

The smooth nature of the condom layer 52 is similar to the aforementioned design, and thus a molten resin can be deposited to create the ruler form 56. The amount of resin deposited on the ruler form 56 allows the manufacturer to manipulate its thickness from a visible standpoint, yet virtually adds no substantial girth to the condom wall. Correspondingly, layer(s) of resin may be applied to form embossed areas that protrude on the lateral side of the condom 50. U.S. Pat. No. 3,616,152 to Chandler outlines a viable method of resin film deposition on a substrate.

The resulting resin deposit will show peaks 60, adjacent to valleys 62, on the design of the ruler 56. This ruler is similar to ruler 20 in FIG. 1. The valleys 62 can potentially extend to the inner layer 52 surface, or they may stop short of contacting the same layer 52. The layer 52 and embossed ruler 56 may then be subjected to an additional dipping step, wherein the outer film 54 is formed as an outer layer of condom 50. In this configuration, the outer film 54 covers the resin deposit in the ruler form 56, while simultaneously conforming to the peaks 60 and valleys 62.

The raised ruler 56 connects to the surrounding outer film 54 by smooth transitions 64. Due to the thickness of the ruler 56, the condom 50 becomes slightly stiffer at its lateral side. Thus, the precision of measurement is maintained by the prevention of a longitudinal pull on the condom that could potentially deform the ruler 56.

The condom 50 has an added anti-slip feature. By controlling the film ruler 56 for elasticity, an optimal constriction of the condom around the penis (even in a flaccid state) prevents the condom from slipping off prematurely. This problem is frequently encountered before and after a full erection of the penis.

The silicon film ruler 56 lodged within the condom 50 is pliable enough to roll the condom walls into the dense form required for commercial distribution, yet it is simultaneously stiff enough to resist a longitudinal compression. This ultimately prevents the condom from collapsing during operation; a mishap that could lead to the failure of contraception.

The niche 24 of condom 10 in FIG. 3 and the embossed ruler 56 of condom 50 in FIG. 4 may be combined so that a lengthwise protrusion of the sheath extends for the entire length of the condom. It is also possible to place the indicia on either the inside or outside of the condom; this is achieve by depositing the embossed resin ruler over the inner layer niche.

FIG. 5 shows a condom erectometer 100 according to a third embodiment of the present invention. Here, a complete ruler 120 on a film substrate is buried within the condom 100. As discussed above, the ruler 120 may be placed between two of the multiple dipping steps during the manufacture of the condom 100. The front end of the ruler 120 may be positioned in the condom 100 where the front end of the penis will rest; this allows the wearer to swiftly adjust the zero point of the ruler relative to the specific point of his penis that he desires to measure from, such as external distal end, the erectile corpus cavernosum penis, or the outline of the crown in the glands.

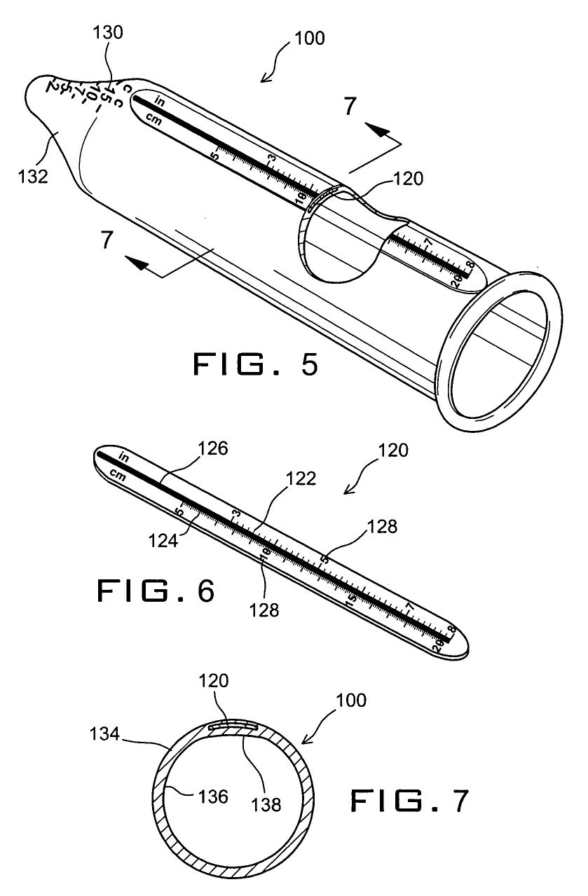

FIG. 6 shows a single film ruler 120 with printed length marks, which may indicate the English system of measurement as shown at 122 starting from a small number such as three to eight or greater inches on one side; the Metric system of measurement as shown at 124 from a selected number range of five to twenty centimeters extends in parallel with the inch marks 122. The aforementioned scale marks are joined by a wider centerline 126 that begins at the front tip of the ruler 120. Numerical characters 128 may be printed proximal to the scale marks in their respective locations. For manufacturing purposes, a roll of web can temporarily hold a series of the ready-made film rulers 120 with their bottom surfaces exposed. To transfer the ink, the rulers 120 may be pressed against the first dipped layer of the condom 100 before the layer is completely dry. The tack of the latex holds the film ruler 120 in place until it is covered by additional dipped layer of the condom 100. An optional cubic centimeter (CC) volume scale 130 is also transferred to the dome area 132 of the condom 100 via the ink print method.

FIG. 7 shows a cross section of the condom 100 wherein the outer surface 134 makes a continuous circle to provide complete smoothness for the user. The inner surface 136 is slightly withdrawn at the side 138 which faces toward the axis of the condom 100; this functions to level the added thickness of the film ruler 120. The shift of the inner side 138 from the circular plane of surface 136 can be manufactured by flattening the mandrel that shapes the condom 100.

Due to the ruler insert, the condom 100 has an added rigidity characteristic that provides enhanced grip onto the lateral side of the penis. The design also adds resistance to prevent an undesirable, lengthwise collapse of the condom walls; this, in effect, improves the effectiveness of the contraceptive device as well as accuracy of length measurement.

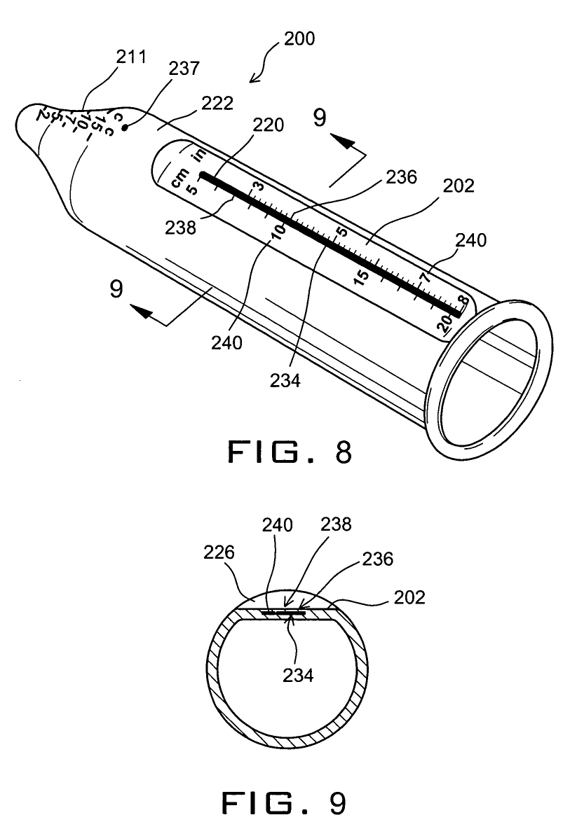

A fourth embodiment of the present invention is illustrated in FIGS. 8 through 11. This variation of the condom 200 is a combination of both the reduced diameter of the inner side 138 of condom 100 as shown in FIG. 7 and the ink indicia 20 on the surface of the inner layer 42 that is sealed by another layer 54, as shown in FIG. 3. The condom 200 has a slightly wider lengthwise flat wall 202 wherein a flat layer of printed ink can be deposited to make the indicia 220, which include a centerline 234, scales 236, 238 and length marks 240. See FIG. 9. FIG. 10, an elevated side view, effectively illustrates the method in which the flat wall 202 merges into the surrounding condom walls. The leading edge 226 of the flat wall 202 bends toward the outer portion 212 of the condom sheath in a fluid line that then merges into the dome section 212 of the condom 200.

The opposite end of the flat wall 202 creates a trailing edge 228 that can be identically shaped to match the leading edge 226. Slacks from the opposite edges 226, 228 strategically absorb potential pulls on the condom walls; this prevents the incidental variation in condom length during the initial manipulation over the penis while still providing an accurate length that is visible through the condom walls.

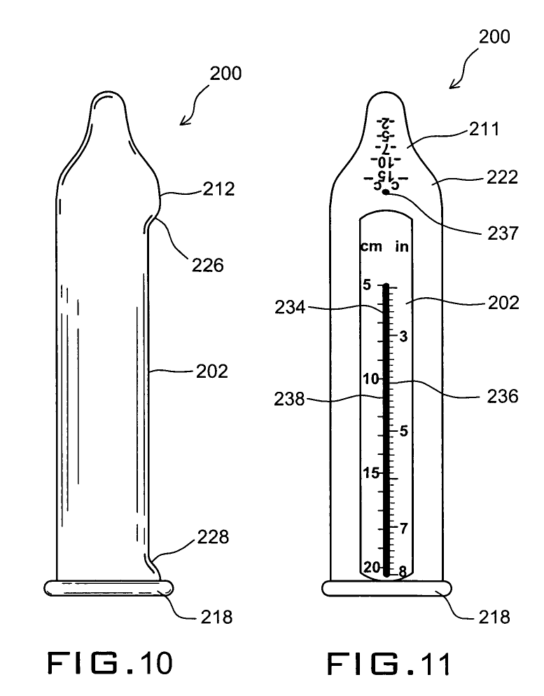

FIG. 11 clearly portrays the simplicity and user-friendliness of the erectomer within the condom 200. The flat section 202 has the scale indicia 220, which is clearly visible to the user when turned to face the top of the penis. The length marks may indicate the English measurement as shown at 236 from a separate zero point 237 to eight or greater inches at one side. On the other side, length marks indicate the Metric system of measurement as shown at 238 from the same zero point 237 to twenty centimeters, and they extend in parallel with the aforementioned inch marks 236. The two sets of scale marks are joined by a wide centerline 234 that may start at the 5 cm mark and end short of a condom ring 218. A scale 211 that measures volume in cubic centimeters may also be printed on the dome area 222 with the number orientation upside down to correctly indicate the amount of ejaculate at the time of disposal.

Advantages of earlier embodiments are retained in the condom 200; this includes the anti-slip feature provided by selected reduction in diameter of the condom walls, as well as the possibility of a prolonged erection at the user's discretion. If combined with the previously mentioned baggy configuration, the condom 200 may become a versatile contraceptive aid to enhance protected intercourse for both sexes.

While the presently preferred form of the condom erectometer has been shown and described, and several modifications thereof discussed, persons skilled in this art will readily appreciate that various additional changes and modifications may be made without departing from the spirit of the invention, as defined and differentiated by the following claims.

For example, the embossed ruler 56 of the condom 50 in the second embodiment may replace the flat indicia 220 of condom 200 in the fourth embodiment within the scope of the claimed invention. Also, the indicia of length marks may include the Metric system of measurement units on one side, and an alternative regional unit (other than the English system) on the other side, which would effectively fit the diverse needs of different world regions. The indicia expressed in Braille points or raised letters naturally fit the principle of the present invention as well.

1. A condom with an erectometer for measuring a longitudinal extent of the erection of a male sexual organ as it is under a contraceptive protection comprising:
a generally tubular sheath formed by at least an inner layer and an outer layer of an elastic material, the sheath having a closed end with a dome and an opposite open end;
a lengthwise protrusion of the sheath extending longitudinally thereof; and
indicia sandwiched between the inner and outer layers of the sheath for indicating a dual length unit having two different scale marks positioned side by side, the indicia extending approximately centrally of the lengthwise protrusion and being visible through the outer layer.

2. The condom with an erectometer of claim 1, wherein the elongated protrusion of the sheath is formed by a niche on the inner layer of the sheath, the niche extending from the dome to a point short of the open end and being shifted toward the central axis of the tubular sheath.

3. The condom with an erectometer of claim 2, wherein the niche of the inner condom layer combined with the indicia provides a local constriction for the user to selectively apply to his sexual organ in order to suppress the ejaculation and prolong the same.

4. The condom with an erectometer of claim 2, wherein the indicia are formed with the use of ink for printing at least a start mark, names of units, centerline, scales and numeral length marks.

5. The condom with an erectometer of claim 1, wherein the indicia are formed in part by depositing resin in an embossed form of ruler on a continuously circumferential section of the inner layer of the sheath and the elongated protrusion of the sheath is formed by raised areas of the outer layer of the sheath extending away from the central axis of the sheath.

6. The condom with an erectometer of claim 1, wherein the elongated protrusion of the sheath is formed in combination of a niche on the inner layer of the sheath shifted toward the central axis of the sheath and raised areas of the outer layer of the sheath extending away from the central axis of the sheath, the protrusion extending from the dome to a point short of the open end and wherein the indicia are formed in part by depositing resin in an embossed form of ruler over the inner layer niche.

7. The condom with an erectometer of claim 1, wherein the sheath is transparent or semitransparent with colors including primary and non-primary colors.

8. The condom with an erectometer of claim 1, further comprising locally extended walls of the sheath with ribbed or studded regions along the internal and external surfaces of the condom.

9. The condom with an erectometer of claim 1, wherein the indicia are formed by printing with the use a rotary press.

10. The condom with an erectometer of claim 1, wherein the indicia are formed by printing with the use a rotogravure press.

11. The condom with an erectometer of claim 1, wherein the indicia further comprise a print of volume scale on the dome of the sheath to indicate the amount of ejaculation of a condom user.

12. The condom with an erectometer of claim 1, wherein the elongated protrusion provides a stiff support for isolating the indicia from dimensional changes of the sheath due to its elasticity to maintain the trueness of length measurement during manipulation of the condom on the erect sexual organ.

13. A condom with an erectometer for measuring a longitudinal extent of the erection of a male reproductive organ as it is under a contraceptive protection comprising:
a generally tubular sheath formed by at least an inner layer and an outer layer of an elastic material, the sheath having a closed end with a dome and an opposite open end;
a lengthwise protrusion of the sheath extending longitudinally thereof; and
a ruler insert sandwiched between the inner and outer layers of the sheath having indicia of a dual length unit having two different scale marks positioned side by side, the indicia extending approximately centrally of the elongated protrusion and being visible through the outer layer.

14. The condom with an erectometer of claim 13, wherein the elongated protrusion of the sheath is formed by a partial withdrawal of the inner layer toward the central axis of the sheath to provide a flattened wall section relative to the circumferential wall areas of the rest of the inner layer, the flattened wall section extending from the dome to a point short of the open end.

15. The condom with an erectometer of claim 13, wherein the indicia are formed by printing on the ruler insert at least names of units, a centerline, scales and numeral length marks.

16. The condom with an erectometer of claim 13, wherein the ruler insert provides a stiff support for isolating the indicia from dimensional changes of the sheath due to its elasticity to maintain the trueness of length measurement during manipulation of the condom on the erect sexual organ.

17. The condom with an erectometer of claim 16, wherein the flattened wall section of the inner condom layer combined with the ruler insert provides a local constriction for the user to selectively apply to his sexual organ in order to suppress the ejaculation and prolong the same.

18. A condom with an erectometer for measuring a longitudinal extent of the erection of a male sexual organ as it is under a contraceptive protection comprising:
a generally tubular sheath formed by at least an inner layer and an outer layer of an elastic material, the sheath having a closed end with a dome and an opposite open end;
a flattened wall section of the sheath relative to the circumferential areas of the rest of the sheath, the flattened wall section extending longitudinally of the sheath from the dome to a point short of the open end; and
indicia sandwiched between the inner and outer layers of the sheath for indicating a dual length unit having two different scale marks positioned side by side, the indicia extending approximately centrally of the elongated protrusion and being visible through the outer layer.

19. The condom with an erectometer of claim 18, further comprising locally extended walls of the sheath with ribbed or studded regions along the internal and external surfaces of the condom.

20. The condom with an erectometer of claim 18, wherein the indicia are formed with the use of ink for printing on the inner layer at the flattened wall section of the sheath at least a start mark, names of units, centerline, scales and numerical length marks.
